(19) United States
(12) Patent Application Publication
(10) Pub. No.: US 2024/0423823 A1
(43) Pub. Date: Dec. 26, 2024

(54) ASYMMETRIC ADHESIVE PACKAGING ENCLOSURE FOR EASE OF MATERIAL REMOVAL

(71) Applicant: Hollister Incorporated, Libertyville, IL (US)

(72) Inventors: Adrian P. Defante, Chicago, IL (US); Mark W. Jockel, Vernon Hills, IL (US)

(21) Appl. No.: 18/698,535

(22) PCT Filed: Nov. 16, 2022
(86) PCT No.: PCT/US2022/050041

§ 371 Date: Apr. 4, 2024

Related U.S. Application Data
(60) Provisional application No. 63/284,682, filed on Dec. 1, 2021.

(51) Int. Cl. A61F 5/443; A61F 5/445

(52) U.S. Cl. CPC A61F 5/443; A61F 5/445

(57) ABSTRACT

A packaging enclosure for an ostomy appliance includes a body having a base and an upstanding sidewall integral and contiguous with the base and having a peripheral edge opposite the base. The base and sidewall define a storage region for the ostomy appliance. The base and sidewall are formed from a first material. A cover element extends over the storage region and is formed from a second material. The second material has a higher affinity for the ostomy appliance than the first material affinity for the ostomy appliance. An ostomy appliance and packaging enclosure is also enclosed.

### BACKGROUND

The following description relates generally to a packaging enclosure and more particularly to a packaging enclosure for an adhesive ostomy appliance.

Adhesive appliances, such as barrier rings are packaged and stored in enclosures to maintain the cleanliness and integrity of the appliance. Barrier rings are formed from a material that is, to some extent, inherently adhesive or sticky. A typical enclosure uses a molded clamshell or clamshell-like enclosure or package to secure and maintain the appliance (barrier ring). The barrier ring has release liners, which are protective coverings, on one or both sides of the barrier ring as it is stored in the enclosure.

To use the barrier ring, the enclosure is opened and the barrier ring is removed from the enclosure. The release liners are removed, and the barrier ring is stretched or otherwise contoured to the desired shape.

While current package configurations function well to protect the barriers in the enclosure, one issue with such enclosures is that a user has to remove the barrier from the enclosure and then remove the release liners from one or both sides of the barrier prior to use. This can be problematic in an ostomy care setting in which a user is attempting to remove and apply the barrier to the area around the user's stoma.

Accordingly, it is desirable to provide an ostomy barrier ring package that permits readily removing the appliance in a ready to use state. Desirably, such a package reduces the handling needed by the user in preparing and applying the barrier ring.

### SUMMARY

In an aspect, a packaging enclosure for an ostomy appliance such as a barrier ring that is adhesive, includes a body having a base and an upstanding sidewall integral and contiguous with the base. The upstanding side wall has a peripheral edge opposite the base. The base and sidewall define a storage region for the barrier ring. The base and sidewall are formed from a first material.

A cover element extends over storage region on the sidewall peripheral edge. The cover element is formed from a second material different from the first material. The second material has a higher affinity for the ostomy appliance, e.g., the barrier ring, than the first material affinity for the ostomy appliance. In this manner, as the cover is removed from the body, the ostomy appliance, e.g., the barrier ring, remains affixed to the cover.

In an embodiment, the body further includes a pedestal formed therein that extends upwardly from the base. In embodiments, the cover element is sealed to the sidewall peripheral edge. The sidewall can be circular in cross-section.

In an embodiment, to further facilitate use, the cover element can include a grasping region that extends from an edge thereof.

In an embodiment, the body can be formed of a polymeric material such as polyethylene (PE), polyethylene terephthalate (PET), polypropylene, polyester, polyimide, polyoxymethylene, organosiloxane, or cellulosic or paper-based materials or combinations thereof. The cover likewise can be formed of a polymeric material, such as polyethylene (PE), polyethylene terephthalate (PET), polypropylene, polyester, polyimide, polyoxymethylene, organosiloxane, or cellulosic or paper-based materials or combinations thereof. Other suitable materials will be recognized by those skilled in the art.

In another aspect, an ostomy appliance and packaging enclosure includes an ostomy appliance, such as a barrier ring, which appliance has first and second surfaces. The ostomy appliance may be formed from an adhesive material.

In embodiments, a packaging enclosure includes a body having a base and an upstanding sidewall integral and contiguous with the base. The body has a peripheral edge opposite the base. The base and sidewall define a storage region for the appliance. The base and sidewall formed from a first material.

A cover element is formed form a second material different from the first material. The ostomy appliance is positioned in the storage region with the first surface positioned on the base and the cover element is positioned on the upstanding sidewall peripheral wall and in contact with the ostomy appliance second surface. The second material has a higher affinity for the ostomy appliance than the first material affinity for the ostomy appliance, such that when the cover is removed from the body, the ostomy appliance, e.g., the barrier ring, remains affixed to the cover. The cover can be sealed to the upstanding side wall.

In embodiments, the barrier can be formed from a hydrocolloid material. The barrier ring material can further include ceramide. The packaging enclosure body can be formed of a polymeric material, such as polyethylene (PE), polyethylene terephthalate (PET), polypropylene, polyester, polyimide, polyoxymethylene, organosiloxane, or cellulosic or paper-based materials or combinations thereof. The cover likewise can be formed of a polymeric material, such as polyethylene (PE), polyethylene terephthalate (PET), polypropylene, polyester, polyimide, polyoxymethylene, organosiloxane, or cellulosic or paper-based materials or combinations thereof. Other suitable materials will be recognized by those skilled in the art.

Other objects, features, and advantages of the disclosure will be apparent from the following description, taken in conjunction with the accompanying sheets of drawings, wherein like numerals refer to like parts, elements, components, steps, and processes.

### BRIEF DESCRIPTION OF THE DRAWINGS

FIG. 1 is a perspective view of an example of an ostomy barrier ring;

FIG. 2 is a side view of a packaging enclosure with a cover according to an embodiment;

FIG. 3 illustrates the barrier ring of FIG. 1 in the packaging enclosure of

FIG. 2;

FIG. 4 shows the barrier ring of FIG. 3 as it is being removed from the enclosure;

FIG. 5 illustrates the barrier ring removed from the enclosure with the packaging enclosure cover thereon; and

FIG. 6 shows the enclosure with the barrier ring removed.

### DETAILED DESCRIPTION

While the present disclosure is susceptible of embodiment in various forms, there is shown in the drawings and will hereinafter be described one or more embodiments with the understanding that the present disclosure is to be considered illustrative only and is not intended to limit the disclosure to any specific embodiment described or illustrated.

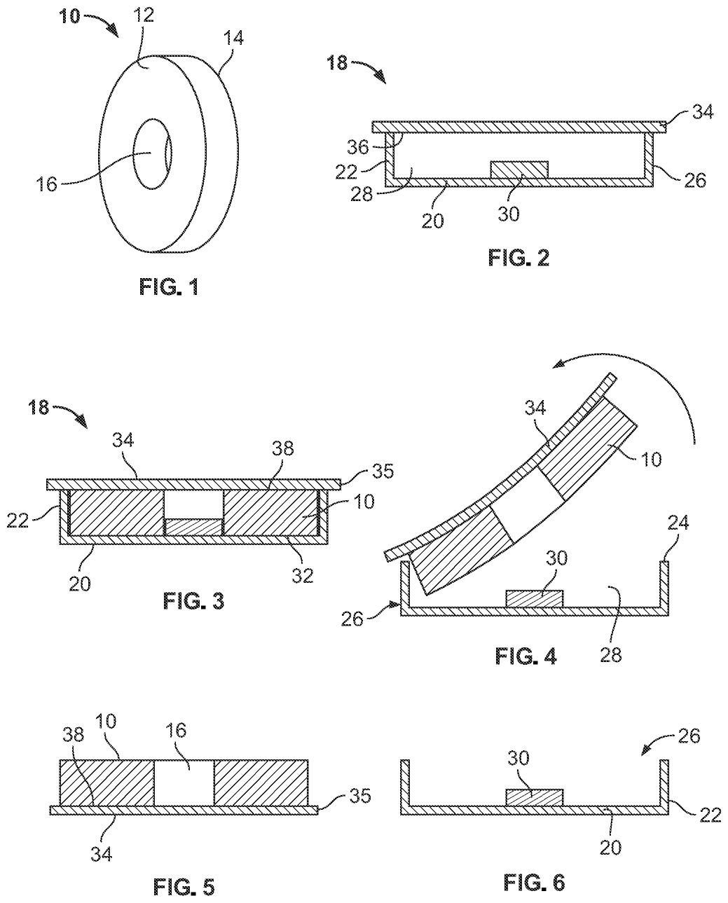

FIG. 1 illustrates an ostomy appliance, such as a barrier ring 10. The barrier ring 10 may be formed from a pressure sensitive adhesive, such as a flexible hydrocolloid material, that may include, for example, ceramide. The barrier ring 10 may have first and second sides 12, 14 and an open central region 16. Referring to FIGS. 2-4, the barrier ring 10 may be packaged and stored in a packaging enclosure 18. In an embodiment, the enclosure 18 may include a base 20 and an upstanding sidewall 22 having a peripheral edge 24. The base 20 and sidewall 22 may define a cup-shaped body 26 having an interior or storage region 28 in which the barrier 10 may be positioned. An upstanding pedestal 30 can be formed in the base 20, such that the open central region 16 of the barrier ring 10 is centered about the pedestal 30. The barrier ring 10 may be positioned in the enclosure 18 with the first side 12 positioned on the base 20 to define a first interface 32.

A cover element 34 may have a barrier ring facing surface 36 that is positioned on the second side 14 of the barrier ring 10 to define a second interface 38. The cover element 34 may extend over the sidewall peripheral edge 24. In embodiments, the cover 34 may be sealed to the peripheral edge 24 to close or seal the enclosure 18. The cover 34 can be formed from a flexible, pliable material, such as a film, that permits the cover 34 to be readily peeled off of the enclosure 18. In embodiments, a portion of an edge of the cover 34 may extend over and beyond the peripheral edge 24 to form a grasping region 35.

To facilitate removal of the barrier ring 10 from the enclosure 18, the materials of the enclosure 18 and the cover 34 may be such that the cover 34 has a higher affinity for the barrier ring 10 material than the barrier ring 10 material has for the enclosure 18 material. That is, the barrier ring 10 may have a greater adherence to the cover 34 than to the enclosure 18. Thus, with the barrier ring 10 stored in the package 18, a user may pull the cover 34 from the enclosure 18, the barrier ring 10 releases from the enclosure 18 and remains affixed to the cover 34. That is, the barrier ring 10 may release from the enclosure 18 and pull away with the cover 34. The user can then readily remove the barrier ring 10 from the cover 34 to use the ring 10.

One barrier 10 material may be a flexible hydrocolloid material, that may include, for example, ceramide. Accordingly, in an embodiment, the body 26 can be formed from a polymeric material, such as polyethylene (PE), polyethylene terephthalate (PET), polypropylene, polyester, polyimide, polyoxymethylene, organosiloxane, or cellulosic or paper-based materials or combinations thereof. Other suitable materials will be recognized by those skilled in the art. The cover 34 can likewise be formed from a polymeric material, such as polyethylene (PE), polyethylene terephthalate (PET), polypropylene, polyester, polyimide, polyoxymethylene, organosiloxane, or cellulosic or paper-based materials or combinations thereof. Other suitable materials will be recognized by those skilled in the art. In such a configuration, the cover 34 may have a greater affinity for the barrier ring 10 than the body 26 affinity for the barrier ring 10. As such, as the cover 34 is removed from the body 26, the barrier ring 10 may remain adhered to the cover 34 for ease of handling and use by a user.

All patents referred to herein, are hereby incorporated herein in their entirety, by reference, whether or not specifically indicated as such within the text of this disclosure.

In the present disclosure, the words “a” or “an” are to be taken to include both the singular and the plural. Conversely, any reference to plural items shall, where appropriate, include the singular. In additions, various features described with respect to any of the embodiments above may be used together, implemented in, or replace features in any of the other embodiments described above.

From the foregoing it will be observed that numerous modifications and variations can be effectuated without departing from the true spirit and scope of the novel concepts of the present invention. It is to be understood that no limitation with respect to the specific embodiments illustrated is intended or should be inferred. The disclosure is intended to cover by the appended claims all such modifications as fall within the scope of the claims.

1. A packaging enclosure for an ostomy appliance, the enclosure comprising:
a body having a base and an upstanding sidewall integral and contiguous with the base and having a peripheral edge opposite the base, the base and sidewall defining a storage region, the base and sidewall formed from a first material; and
a cover element, the cover element formed from a second material,
wherein the second material has a higher affinity for the ostomy appliance than the first material affinity for the ostomy appliance.

2. The packaging enclosure of claim 1 further including a pedestal formed in the body extending upwardly from the base.

3. The packaging enclosure of claim 1, wherein the cover element is sealed to the sidewall peripheral edge.

4. The packaging enclosure of claim 1, wherein the sidewall is circular in cross-section.

5. The packaging enclosure of claim 1, wherein the cover element includes a grasping region extending from an edge thereof.

6. The packaging enclosure of claim 1, wherein the body is formed of polyethylene (PE), polyethylene terephthalate (PET), polypropylene, polyester, polyimide, polyoxymethylene, organosiloxane, or cellulosic or paper-based materials or combinations thereof.

7. The packaging enclosure of claim 1, wherein the cover is formed of polyethylene (PE), polyethylene terephthalate (PET), polypropylene, polyester, polyimide, polyoxymethylene, organosiloxane, or cellulosic or paper-based materials or combinations thereof.

8. The packaging enclosure of claim 6, wherein the cover is formed of polyethylene (PE), polyethylene terephthalate (PET), polypropylene, polyester, polyimide, polyoxymethylene, organosiloxane, or cellulosic or paper-based materials or combinations thereof.

9. An ostomy appliance and packaging enclosure, comprising:
an ostomy appliance, the ostomy appliance having first and second surfaces, the ostomy appliance formed from an adhesive material;
a packaging enclosure comprising:
a body having a base and an upstanding sidewall integral and contiguous with the base and having a peripheral edge opposite the base, the base and sidewall defining a storage region, the base and sidewall formed from a first material; and
a cover element, the cover element formed from a second material,
wherein the ostomy appliance is positioned in the storage region with the first surface positioned on the base,
wherein the cover element is positioned on the upstanding sidewall and in contact with the ostomy appliance second surface, and
wherein the second material has a higher affinity for the ostomy appliance than the first material affinity for the ostomy appliance.

10. The ostomy appliance and packaging enclosure of claim 9, wherein the ostomy appliance remains affixed to the cover when the cover is removed from the packaging enclosure.

11. The ostomy appliance and packaging enclosure of claim 9, wherein the cover is sealed to the upstanding side wall.

12. The ostomy appliance and packaging enclosure of claim 9, wherein the body is formed of polyethylene (PE), polyethylene terephthalate (PET), polypropylene, polyester, polyimide, polyoxymethylene, organosiloxane, or cellulosic or paper-based materials or combinations thereof.

13. The ostomy appliance and packaging enclosure of claim 9, wherein the cover is formed of polyethylene (PE), polyethylene terephthalate (PET), polypropylene, polyester, polyimide, polyoxymethylene, organosiloxane, or cellulosic or paper-based materials or combinations thereof.

14. The ostomy appliance and packaging enclosure of claim 12, wherein the cover is formed of polyethylene (PE), polyethylene terephthalate (PET), polypropylene, polyester, polyimide, polyoxymethylene, organosiloxane, or cellulosic or paper-based materials or combinations thereof.

15. The ostomy appliance and packaging enclosure of claim 9, wherein the ostomy appliance is a barrier ring.

16. The ostomy appliance and packaging enclosure of claim 15, wherein the barrier ring is formed from a hydrocolloid material.

17. The ostomy appliance and packaging enclosure of claim 16, wherein the barrier ring material further includes ceramide.
